(19) United States
(12) Patent Application Publication
(10) Pub. No.: US 2026/0283683 A1
(43) Pub. Date: Sep. 24, 2026

(54) SYSTEMS AND METHODS FOR ENERGY DELIVERY

(71) Applicant: St. Jude Medical, Cardiology Division, Inc., St. Paul, MN (US)

(72) Inventors: Isaac REMER, Richfield, MN (US); Greg Olson, Elk River, MN (US); Derek Sutermeister, St. Paul, MN (US)

(21) Appl. No.: 19/472,634

(22) PCT Filed: Apr. 4, 2024
(86) PCT No.: PCT/US24/22927

§ 371 Date: Oct. 3, 2025

Related U.S. Application Data
(60) Provisional application No. 63/457,252, filed on Apr. 5, 2023.
(60) Provisional application No. 63/534,958, filed on Aug. 28, 2023.
(60) Provisional application No. 63/534,965, filed on Aug. 28, 2023.

(51) Int. Cl. A61B 18/14; A61B 18/00; A61B 18/12; A61B 18/16

(52) U.S. Cl. CPC A61B 18/1492; A61B 18/1206; A61B 18/16; A61B 2018/00351; A61B 2018/00577

(57) ABSTRACT

A catheter assembly is provided. The catheter assembly includes a catheter including at least one lesion generating electrode, the at least one lesion generating electrode configured to be positioned within a patient, and at least one return array configured to be positioned within the patient and remote from the at least one lesion generating electrode, the at least one return array including at least one return electrode, wherein the catheter assembly is configured to apply energy between i) the at least one lesion generating electrode and ii) a return patch and the at least one return electrode to generate lesions proximate the at least one lesion generating electrode.

### CROSS-REFERENCE TO RELATED APPLICATIONS

This application claims priority to U.S. Provisional Patent Application No. 63/457,252 filed on Apr. 5, 2023, U.S. Provisional Patent Application No. 63/534,958, filed on Aug. 28, 2023, and U.S. Provisional Patent Application No. 63/534,965, filed on Aug. 28, 2023, all of which are incorporated by reference herein in their entirety.

### FIELD OF THE DISCLOSURE

The present disclosure relates generally to tissue ablation systems. In particular, the present disclosure relates to systems for reducing skeletal muscle recruitment.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to cause cell death (e.g., via tissue apoptosis or necrosis) in cardiac tissue to correct conditions such as atrial arrhythmia (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a non-thermal ablation technique that involves applying strong electric-fields that induce pore formation in the cellular membrane. The electric field may be induced by applying a relatively short duration pulse which may last, for instance, from a nanosecond to several milliseconds. Such a pulse may be repeated to form a pulse train. When such an electric field is applied to tissue in an in vivo setting, the cells in the tissue are subjected to trans-membrane potential, which opens the pores on the cell wall. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

Irreversible electroporation, also referred to as pulsed field ablation (PFA), is an emerging technology with potential advantages over other types of ablation. The mechanism of lesion formation in PFA is a function of electric field exposure that breaks down cell membrane permeability, leading to cell death. PFA employs (most commonly) trains of bipolar and biphasic high voltage and very-short duration pulses that result in destabilization of the cellular membranes (formation of pores in the cytoplasmic membrane) and cell death. This method has several potential advantages for ablation of cardiac arrhythmias, including higher selectivity to myocardial tissue and smaller thermal effect.

At least some known PFA systems are single catheter PFA systems. These include monopolar catheters that deliver energy from a catheter to a single back patch, and bipolar catheters that delivery energy between electrodes contained on the same catheter. At least some monopolar catheter applications may, in some situations, be accompanied by significant skeletal muscle recruitment. At least some bipolar catheter applications may minimize or eliminate skeletal muscle recruitment, but may, in some situations, have difficulty achieving the same lesion depth as monopolar catheter applications and may, in some situations, result in bubble formation.

Accordingly, it would be desirable to provide a catheter system that combines the advantages of monopolar and bipolar catheters, while avoiding potential disadvantages of monopolar and bipolar catheters.

### BRIEF SUMMARY OF THE DISCLOSURE

In one aspect, a catheter assembly is provided. The catheter assembly includes a catheter including at least one lesion generating electrode, the at least one lesion generating electrode configured to be positioned within a patient, and at least one return array configured to be positioned within the patient and remote from the at least one lesion generating electrode, the at least one return array including at least one return electrode, wherein the catheter assembly is configured to apply energy between i) the at least one lesion generating electrode and ii) a return patch and the at least one return electrode to generate lesions proximate the at least one lesion generating electrode.

In another aspect, an ablation system is provided. The ablation system includes a generator, and a catheter assembly coupled to the generator. The catheter assembly includes a catheter including at least one lesion generating electrode, the at least one lesion generating electrode configured to be positioned within a patient, and at least one return array configured to be positioned within the patient and remote from the at least one lesion generating electrode, the at least one return array including at least one return electrode, wherein the generator is configured to apply energy between i) the at least one lesion generating electrode and ii) a return patch and the at least one return electrode to generate lesions proximate the at least one lesion generating electrode.

In yet another aspect, an ablation system is provided. The ablation system includes a generator, a catheter comprising at least one lesion generating electrode, the at least one lesion generating electrode configured to be positioned within a patient, and a plurality of return patches configured to positioned on the exterior of the patient, wherein the generator is configured to apply energy between the at least one lesion generating electrode and the plurality of return patches to generate lesions proximate the at least one lesion generating electrode.

In yet another aspect, an ablation system is provided. The ablation system includes a generator, and a plurality of electrodes coupled to the generator, wherein the ablation system is configured to apply energy between a first electrode of the plurality of electrodes and a first subset of the plurality of electrodes to generate a first lesion proximate the first electrode, and subsequently apply energy between a second electrode of the plurality of electrodes and a second subset of the plurality of electrodes to generate a second lesion proximate the second electrode, wherein the first and second lesions at least partially overlap.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIG. 2 is a simplified schematic diagram of one embodiment of a catheter assembly for use with an ablation system.

FIG. 3 is a simplified schematic diagram of another embodiment of a catheter assembly for use with an ablation.

FIG. 4 is a simplified schematic diagram of one embodiment of a return array.

FIG. 5 is a simplified schematic diagram of another embodiment of a return array.

FIG. 6 is a simplified schematic diagram of another embodiment of a return array.

FIG. 7A is a simplified schematic diagram of one embodiment of a return array assembly.

FIG. 7B is a simplified schematic diagram of the return array assembly shown in FIG. 7A slid over an introducer that is used to position a lesion generating catheter.

FIG. 8 is a schematic diagram of one embodiment of a patch arrangement.

### DETAILED DESCRIPTION OF THE DISCLOSURE

Systems and methods for a catheter assembly are provided. The catheter assembly includes a catheter including at least one lesion generating electrode, the at least one lesion generating electrode configured to be positioned within a patient, and at least one return array configured to be positioned within the patient and remote from the at least one lesion generating electrode, the at least one return array including at least one return electrode, wherein the catheter assembly is configured to apply energy between i) the at least one lesion generating electrode and ii) a return patch and the at least one return electrode to generate lesions proximate the at least one lesion generating electrode.

Although at least some embodiments of the present disclosure are described with respect to pulmonary vein isolation (PVI), it is contemplated that the described features and methods of the present disclosure as described herein may be incorporated into any number of systems and any number of applications as would be appreciated by one of ordinary skill in the art based on the disclosure herein.

Further, although at least some embodiments of the present disclosure are described with respect to electroporation applications that apply electric fields between electrodes, it is contemplated that the described features and methods may be utilized in other energy delivery applications. For example, in some embodiments, the features and methods describe herein are used in radio frequency (RF) applications that use electrical energy to thermally ablate tissue.

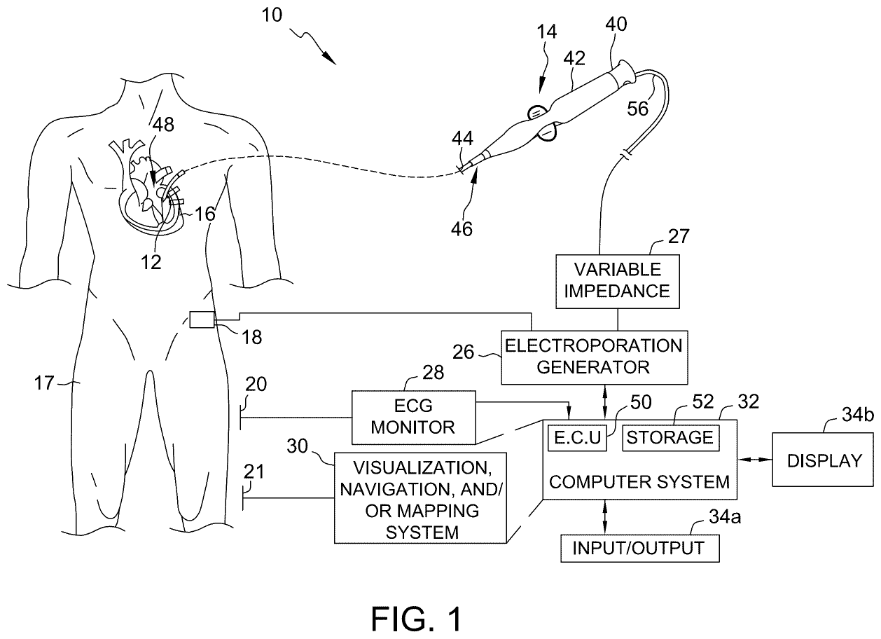

FIG. 1 is a block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

System 10 may be used for pulsed field ablation (PFA) (also referred to as irreversible electroporation (IRE)) to destroy tissue. In particular, system 10 may be used for electroporation-induced primary apoptosis therapy, which refers to the effects of delivering electrical current in such a manner as to directly cause an irreversible loss of plasma membrane (cell wall) integrity leading to its breakdown and cell apoptosis. This mechanism of cell death may be viewed as an “outside-in” process, meaning that the disruption of the outside wall of the cell causes detrimental effects to the inside of the cell. Typically, for classical plasma membrane electroporation, electric current is delivered as a pulsed electric field in the form of short-duration pulses (e.g., having a 0.1 to 20 millisecond (ms) duration) between closely spaced electrodes capable of delivering an electric field strength of about 0.1 to 1.0 kilovolts/centimeter (kV/cm). System 10 may be used, for example, for high output (e.g., high voltage and/or high current) PFA procedures. In some particular embodiments, system 10 is configured to deliver a PFA signal having a relatively high voltage and low pulse duration.

In one embodiment, all electrodes of the catheter deliver an electric current simultaneously. Alternatively, in other embodiments, stimulation is delivered between pairs of electrodes on the catheter. Delivering electric current simultaneously using a plurality of electrodes may facilitate creating a sufficiently deep lesion for electroporation. To facilitate switching between i) activating electrodes simultaneously to deliver energy and ii) activating electrodes to sense signals (e.g., independent from one another), the electrodes may be switchable between being connected to a 3D mapping system and being connected to EP amplifiers.

It should be understood that while the energization strategies are described as involving DC pulses, embodiments may use variations and remain within the spirit and scope of the disclosure. For example, exponentially-decaying pulses, exponentially-increasing pulses, and combinations may be used. Further, in some embodiments, AC pulses may be used. Also, as noted above, in some embodiments, RF energy delivery may be used.

Further, it should be understood that the mechanism of cell destruction in PFA is not primarily due to heating effects, but rather to cell membrane disruption through application of a high-voltage electric field. Thus, PFA facilitates reducing thermal effects of ablation. This “cold therapy” thus has desirable characteristics. However, as noted above, the system and methods described herein may also be used with RF applications.

With this background, and now referring again to FIG. 1, system 10 includes a catheter electrode assembly 12 including at least one catheter electrode. Electrode assembly 12 is incorporated as part of a medical device such as a catheter 14 for PFA therapy of tissue 16 in a body 17 of a patient. In the illustrative embodiment, tissue 16 includes heart or cardiac tissue. It should be understood, however, that embodiments may be used to conduct PFA therapy with respect to a variety of other body tissues.

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode. Further, the patches may be located at any suitable location. For example, one or more patches may be located on a lower back of the patient, and centered relative to the spine of the patient.

In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with a PFA energization strategy, which may be predetermined or may be user-selectable. For PFA-induced primary apoptosis therapy, generator 26 may be configured to produce an electric current that is delivered via electrode assembly 12 as a pulsed electric field in the form of short-duration DC pulses (e.g., a nanosecond to several milliseconds duration, a 0.1 to 20 ms duration, or any duration suitable for electroporation) between closely spaced electrodes capable of delivering an electric field strength (i.e., at the tissue site) of about 0.1 to 1.0 kV/cm. The amplitude and pulse duration needed for PFA are inversely related. As pulse durations are decreased, the amplitude must be increased to achieve PFA.

PFA has been shown to be an effective form of ablation for treatment of cardiac arrhythmias, particularly for instantaneous pulmonary vein isolation (PVI). PFA includes delivering high voltage pulses from electrodes disposed on a catheter (e.g., including the basket and/or balloon catheters described herein). In PFA, for example, voltage amplitudes may range from about 300 V to at least 3,200 V (or even as large as on the order as 10,000 V), and pulse widths may from hundreds of nanoseconds to tens of milliseconds.

These electric fields may be applied between adjacent electrodes (in a bipolar approach) or between a one or more electrodes and a return patch (in a monopolar approach). There are advantages and disadvantages to each of these approaches.

For example, regarding lesion contiguity, the monopolar approach has the potential to leave gaps in lesion coverage (referred to as dead zones) between electrodes where the field strength is low or zero, whereas the field strength in the bipolar approach generally prevents dead zones between electrodes.

For lesion size and proximity, the monopolar approach has a wider range of effect, and can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions. However, the monopolar approach may create larger lesions than are necessary, while the lesions generated using the bipolar approach may be more localized.

Due to a wider range of effect, the monopolar approach may cause unwanted skeletal muscle and/or nerve activation. In contrast, the bipolar approach has a constrained range of effect proportional to electrode spacing on the lead, and is less likely to depolarize cardiac myocytes or nerve fibers.

For the monopolar approach, only a single potential is applied in catheter wires and electrodes. Further, because all the electrodes are at the same polarity, the configuration is not susceptible to arcing (e.g., when using the basket and/or balloon catheters described herein). In contrast, for the bipolar approach, the internal architecture of the catheter must be constructed to prevent arcing, as different electrodes are at different potentials.

As there are advantages and disadvantages of both monopolar and bipolar approaches, it would be desirable to provide a PFA system that realizes the advantages of both approaches while avoiding the disadvantages of both approaches.

Referring back to FIG. 1, electroporation generator 26, sometimes also referred to herein as a DC energy source, is a monophasic electroporation generator 26 configured to generate a series of DC energy pulses that all produce current in the same direction. In other embodiments, electroporation generator is biphasic or polyphasic electroporation generator configured to produce DC energy pulses that do not all produce current in the same direction. In some embodiments, electroporation generator 26 is configured to output energy in DC pulses at selectable energy levels, such as fifty joules, one hundred joules, two hundred joules, and the like. Other embodiments may have more or fewer energy settings and the values of the available setting may be the same or different. For successful electroporation, some embodiments utilize the two hundred joule output level. For example, electroporation generator 26 may output a DC pulse having a peak magnitude from about 300 Volts (V) to about 3,200 V at the two hundred joule output level. In some embodiments, the peak magnitude may be even larger (e.g., on the order of 10,000 V). Other embodiments may output any other suitable positive or negative voltage. For example, in some embodiments, the systems and methods described herein may include pulses with amplitudes from about 500 V to about 4,000 V, with pulse widths from about 200 nanoseconds to about 20 microseconds.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

In other embodiments, one or more semiconductor devices in series with catheter 14 may be used to limit arcing. For example, a specifically engineered semiconductor device, adapted from a field effect transistor, could be implemented, the device being a two terminal device capable of acting very quickly to limit current and power. Two of these devices may be used for biphasic energy delivery, while one device may be used for monophasic energy delivery. Commercially available devices are designed for low currents, usually in the milliamp range, but a semiconductor device for use in PFA applications could be engineered by modifying the size and/or dopant concentrations of existing devices. This would facilitate improving patient safety, and would potentially enable using catheters and generators multiple times.

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

In some embodiments, catheter 14 includes a basket catheter assembly having catheter electrodes (not shown in FIG. 1) distributed at the distal end of shaft 44 in a basket structure. Further, as described herein, an inflatable balloon may be contained within the basket structure.

Localization and navigation system 30 may be provided for visualization, mapping and navigation of internal body structures. Localization and navigation system 30 may include conventional apparatus known generally in the art (e.g., the EnSite X™ Mapping System, as generally shown in U.S. Pat. App. Pub. No. 2020/0138334 titled “Method for Medical Device Localization Based on Magnetic and Impedance Sensors”, the entire disclosure of which is incorporated herein by reference). It should be understood, however, that this system is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N.V., the AURORA® system of Northern Digital Inc., commonly available fluoroscopy systems, or a magnetic location system such as the gMPS system from Mediguide Ltd. In this regard, some of the localization, navigation and/or visualization system would involve a sensor be provided for producing signals indicative of catheter location information, and may include, for example one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

To monitor operation of system 10, one or more impedances between catheter electrodes and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and U.S. Patent Application No. 63/027,660, filed on May 20, 2020, all of which are incorporated by reference herein in their entirety.

The systems and methods described herein involve return electrode arrangements that facilitate realizing the benefits of both monopolar and bipolar approaches to PFA. In some embodiments, as described herein, a PFA system includes a lesion targeting array and at least one return electrode array. The at least one return electrode array is positioned remotely from the lesion targeting array, but, like the lesion targeting array, is positioned within the patient. In other embodiments, a PFA system includes a lesion targeting array and a plurality of return patches distributed on the surface of the patient, as described herein. These embodiments facilitate reducing skeletal muscle recruitment by distributing current using the return electrode arrangements described herein.

As described herein, the lesion targeting array includes one or more lesion generating electrodes. Accordingly, in some embodiments, the lesion targeting array includes a plurality of lesion generating electrodes. In such embodiments, a plurality of lesion generating electrodes may be activated together (e.g., in a “ganged” configuration) to function as a single, larger effective electrode. While an individual lesion generating electrode may generate a spot-shaped, or relatively round lesion, activating multiple lesion generating electrodes in unison enables creating lesions with other desired geometries (e.g., a line lesion, a relatively large circular lesion, an elliptical lesion, etc.). Those of skill in the art will appreciate that many different lesion geometries are possible using the implementations described herein.

Further, although some of the embodiments disclosed herein are described in the context of ring electrodes, those of skill in the art will appreciate that the systems and methods described herein may be implemented using any suitable type of electrode. For example, the lesion generating electrode(s) and the return electrode(s) may include ring electrodes, relatively small spot electrodes (e.g., an array of flexible printed electrodes), domed or rounded electrodes, strut or spline electrodes, and/or any other suitable type of electrode.

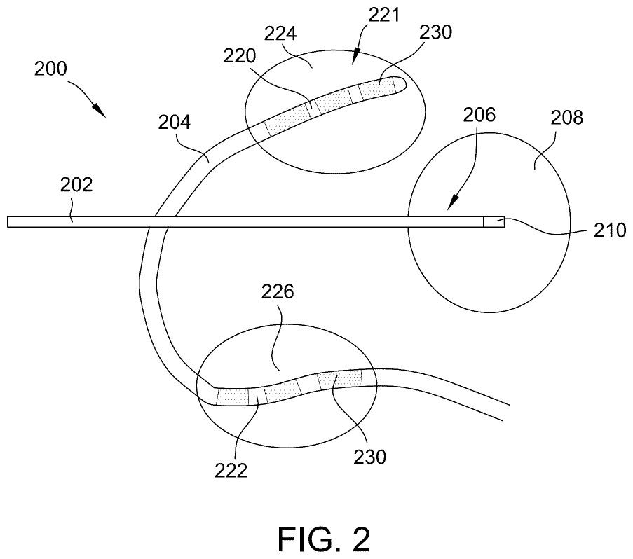

FIG. 2 is a simplified schematic diagram of one embodiment of a catheter assembly 200 for use with an ablation system (such as, for example, system 10 (shown in FIG. 1)). Catheter assembly 200 includes a first catheter 202 and a second catheter 204. A distal end 206 of first catheter 202 is positioned in a lesion targeting location 208 in the patient (i.e., a region where tissue to be ablated is located) and includes at least one lesion generating electrode 210. In this embodiment, second catheter 204 includes a first return array 220 located at a distal end 221 of second catheter 204, and a second return array 222 proximal of first return array 220. First return array 220 is positioned at a first return location 224, and second return array 222 is positioned at a second return location 226. First and second return locations 224 and 226 may be, for example, in a bloodstream of the patient.

First return array 220 and second return array 222 each include at least one return electrode 230. In FIG. 2, each return array 220 and 222 includes three return electrodes 230. Alternatively, each return array 220 and 222 may include any suitable number of electrodes 230. For example, in one embodiment, each return array 220 and 222 includes ten return electrodes 230.

During operation, PFA is accomplished by applying electric fields between at least one lesion generating electrode 210 of first catheter 202 and return electrodes 230 of second catheter 204. Notably, first and second return arrays 220 and 222 have a much larger electrode surface area than at least one lesion generating electrode 210. For example, first and second return arrays 220 and 222 may have a length from approximately 150 millimeters (mm) to 305 mm. Accordingly, when electric fields are applied, current densities at first and second return arrays 220 and 222 are much lower than current densities at least one lesion generating electrode 210. This results in lesions generally being generated at at least one lesion generating electrode 210, but not at first and second return arrays 220 and 222.

This arrangement enables using larger electric fields to create more effective, deeper, larger lesions at lesion targeting location 208, while avoiding unwanted effects at first and second return locations 224 and 226, such as unwanted heating, lesion formation, and/or smooth muscle response. This arrangement also facilitates making first 202 catheter angle agnostic for lesion purposes (i.e., the shape of the resulting legion is substantially unchanged for different orientation angles between the at least one lesion generating electrode 210 and the tissue being ablated).

For example, in one experimental scenario, each of first and second return arrays 220 and 222 included ten return electrodes 230. In this scenario, when generating electric fields between at least one lesion generating electrode 210 and both of return arrays 220 and 222 (i.e., between at least one lesion generating electrode 210 and twenty total return electrodes 230), no lesions were formed at first and second return locations 224 and 226. When generating electric fields between at least one lesion generating electrode 210 and only one of return arrays 220 and 222 (i.e., between at least one lesion generating electrode 210 and ten total return electrodes 230), only superficial lesions were formed at the associated one of first and second return locations 224 and 226.

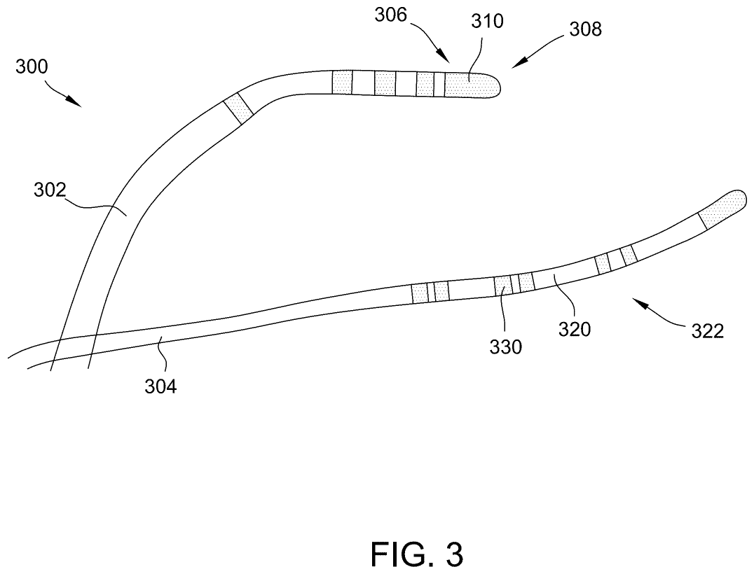

FIG. 3 is a simplified schematic diagram of another embodiment of a catheter assembly 300 for use with an ablation system (such as, for example, system 10 (shown in FIG. 1)). Catheter assembly 300 includes a first catheter 302 and a second catheter 304. A distal end 306 of first catheter 302 is positioned in a lesion targeting location 308 in the patient (i.e., a region where tissue to be ablated is located) and includes at least one lesion generating electrode 310. In this embodiment, second catheter 304 includes a return array 320 located at a distal end 322 of second catheter 304. First return array 320 includes at least one return electrode 330.

In this embodiment, first catheter 302 is positioned, for example, in the left atrium of the patient, and is free to roam with at least one lesion generating electrode 310 to generate lesions as desired.

Second catheter 304 is positioned in the coronary sinus of the patient. Alternatively, the second catheter 304 may be positioned in, for example, the inferior vena cava, the right atrium, or the pericardium of the patient. This positioning of second catheter 304 ensures that return array 320 is proximate tissue that is not susceptible to the PFA energy being applied. Alternatively, or additionally, second catheter 304 may include structural features (described in detail below) that make it physically impossible for at least one return electrode 330 to come in close enough proximity to heart tissue to create a lesion.

During operation, PFA is accomplished by applying electric fields between at least one lesion generating electrode 310 of first catheter 302 and at least one return electrode 330 of second catheter 304. Notably, return array 320 has a much larger electrode surface area than at least one lesion generating electrode 310. Accordingly, when electric fields are applied, current densities at return array 320 are much lower than current densities at least one lesion generating electrode 310. This results in lesions being generated at at least one lesion generating electrode 310, but not at return array 320 By locating at least one return electrode 330 remotely from at least one lesion generating electrode 310, a depth and a size of generated lesions may be easily adjusted by varying the voltage of the applied electric field. Specifically, with this arrangement, lesion depth attained with single voltages may be significantly higher (allowing for use of lower voltages), and microbubble formation may also be reduced.

Although the embodiments shown in FIGS. 2 and 3 show two catheters (e.g., one catheter with electrodes for forming lesions, and another catheter including one or more return arrays), those of skill of art will appreciate that other embodiments are possible. For example, in one embodiment, a single catheter is used, and the one or more return arrays are located sufficiently distal of the electrodes for forming lesions. In another example, three catheters are used, with a first catheter including electrodes for forming lesions, a second catheter including a first return array, and a third catheter including a second return array.

Further, in the embodiments described herein, energy may be delivered between at least one lesion generating electrode (e.g., lesion generating electrode 210 and lesion generating electrode 310) and a combination of i) one or more external patch electrodes (e.g., return electrodes 18, 20, 21) and ii) one or more internal return electrodes (e.g., electrodes in return arrays 220, 222, and 322). That is, the energy delivered from the at least one lesion generating electrode is distributed between i) one or more external return patches and ii) one or more internal electrodes. This focuses the energy at the at least one lesion generating electrode, causing the lesion to be created proximate that electrode, while dissipating the current between multiple returns (e.g., patches and internal electrodes) such that no lesions are generated at the returns. Notably, the internal electrodes may be located on the same catheter as the at least one lesion generating electrode, or on one or more other catheters.

In one example, the lesion generating electrode is a tip electrode on a catheter, and a combination of i) one or more other electrodes on the same catheter and ii) one or more external patch electrodes function as the returns.

In some embodiments, a relatively large, continuous lesion may be generated by sequentially switching which electrodes function as the lesion generating electrode/return electrodes. For example, consider a catheter including a proximal electrode, a distal electrode, and an intermediate electrode positioned between the proximal electrode and the distal electrode. In a first stage, the proximal electrode is set as the lesion generating electrode, and the intermediate electrode and distal electrodes are set as return electrodes (generating a first lesion proximate the proximal electrode). In a second stage, the intermediate electrode is set as the lesion generating electrode, and the proximal and distal electrodes are set as return electrodes (generating a second lesion proximate the intermediate electrode). In a third stage, the distal electrode is set as the lesion generating electrode, and the proximal and intermediate electrodes are set as return electrodes (generating a third lesion proximate the distal electrode). The switching may be accomplished automatically using a suitable controller (e.g., computer system 32).

If the proximal, intermediate, and distal electrodes are located a sufficient distance from one another, the first, second, and third lesions will at least partially overlap with one another, forming a larger single lesion. Notably, this single lesion may be formed with the catheter located at a single position (i.e., the catheter does not need to be moved between multiple different positions to form the lesion).

Although this example uses three electrodes on the same catheter, those of skill in the art will appreciate that this methodology may be applied using any suitable number of electrodes on one or more catheters.

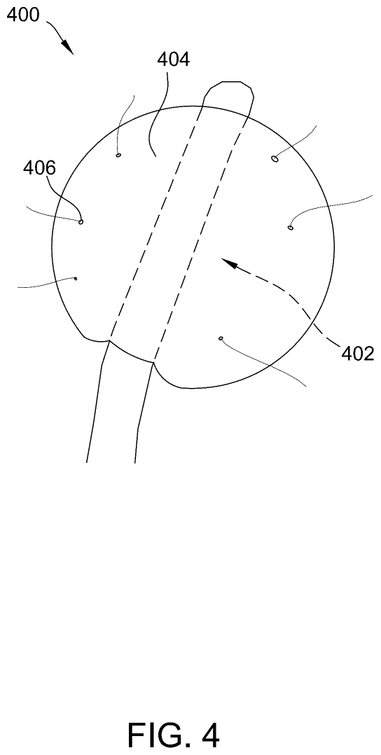

As noted above, the catheter including the one or more return arrays may include a structural feature that make it physically impossible for at least one return electrode to come in close enough proximity to heart tissue to create a lesion. For example, FIG. 4 is a simplified schematic diagram of one embodiment of a return array 400. Return array 400 includes at least one return electrode 402 and a selectively inflatable balloon 404 surrounding at least one return electrode 402. A plurality of irrigation holes 406 are defined in balloon 404. Notably, balloon 404 acts as a buffer or barrier and keeps at least one return electrode 402 at a distance from adjacent tissue, minimizing or eliminating any impact of at least one return electrode 402 on that tissue when electric fields are applied.

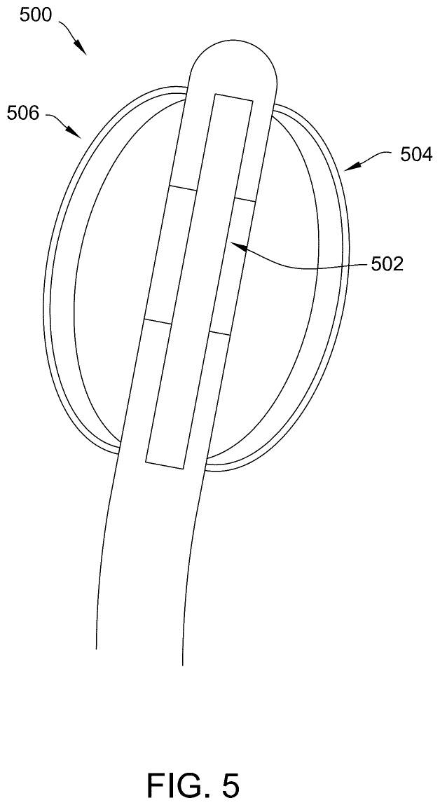

FIG. 5 is a simplified schematic diagram of another embodiment of a return array 500. Return array 500 includes at least one return electrode 502 and a plurality of struts 504 that surround at least one return electrode 502 and form a selectively expandable basket 506. Struts 504 are made of an insulating material, such as nitinol for example. Notably, basket 504 acts as a buffer and keeps at least one return electrode 502 at a distance from adjacent tissue, minimizing or eliminating any impact of at least one return electrode 502 on that tissue when electric fields are applied.

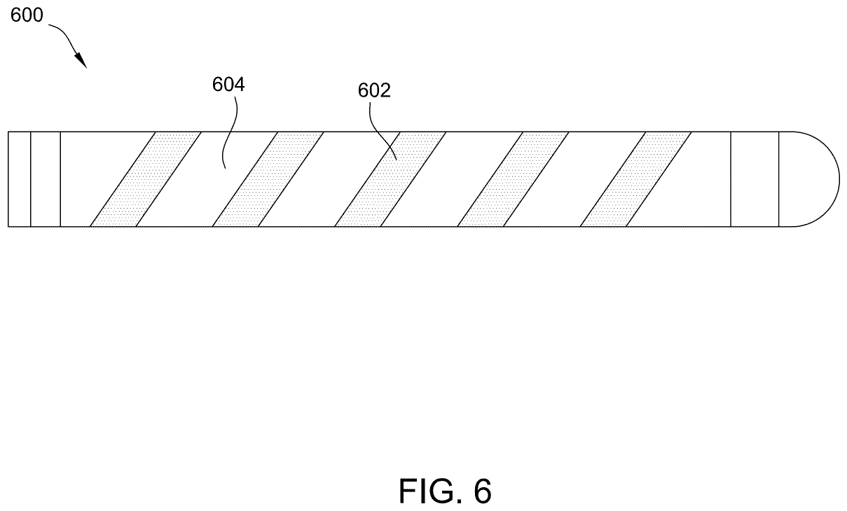

FIG. 6 is a simplified schematic diagram of another embodiment of a return array 600. Return array 600 includes a wrapped electrode 602 that helically wraps around a catheter body 604 multiple times, resulting in a relatively large electrode surface area (and decreased current density).

As those of skill in the art will appreciate, other return array configurations may be used to achieve a relatively large electrode surface area. For example, in some embodiments, the return array includes numerous ring electrodes (e.g., five or more ring electrodes) that are activated together to function as a larger effective electrode (e.g., in a “ganged” configuration). Collectively, the numerous ring electrodes form a relatively large electrode surface area (resulting in decreased current density). In other embodiments, for example, a single elongated electrode and/or a wrapped foil electrode may extend along a portion of the catheter body. Further, in some embodiments, expandable features (e.g., expandable struts, expandable arms, and/or inflatable balloons) may be used to increase the effective electrode surface area.

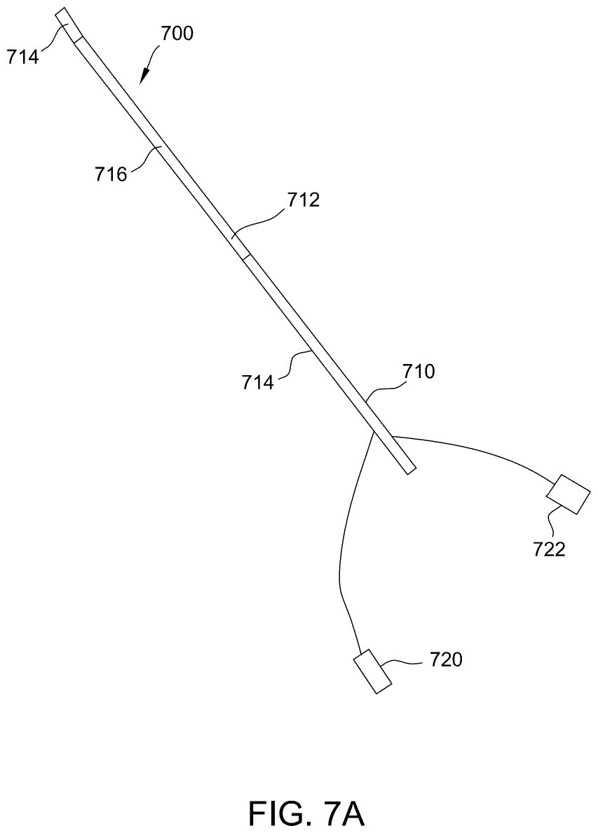

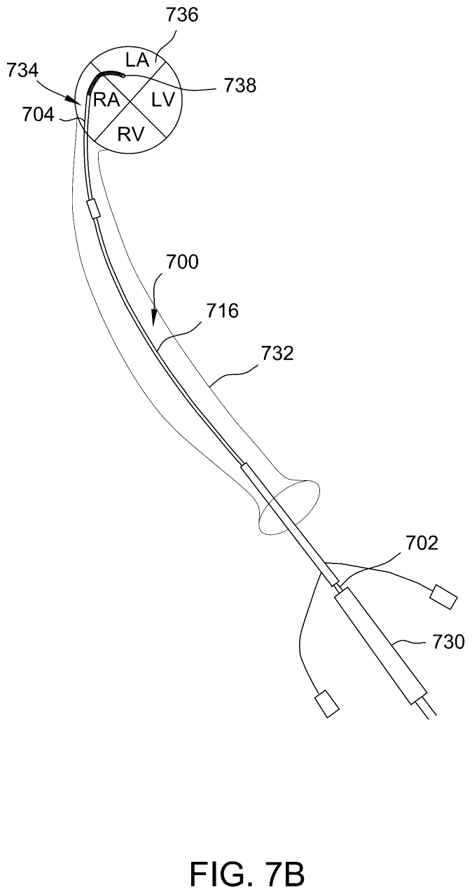

In some embodiments, the return array may be implemented on a separate sleeve that may be slid over or otherwise coupled to the lesion generating catheter and/or an introducer. For example, FIG. 7A is a simplified schematic diagram of one embodiment of a return array assembly 700, and FIG. 7B is a simplified schematic diagram of return array assembly 700 slid over an introducer 702 that is used to position a lesion generating catheter 704.

Return array assembly 700 includes a sleeve 710 sized to slide over introducer 702. Sleeve 710 includes a return array 712 and insulated portions 714. In this embodiment, return array 712 is a single elongated electrode 716. However, other electrode configurations may be used (e.g., multiple ring electrodes, a helically wrapped electrode, coil electrodes, etc.). Return array assembly 700 further includes an electrode connector 720 (e.g., to couple return array 712 to the pulse generator) and a flush port 722 (e.g., to facilitate flushing return array 712 with fluid).

FIG. 7B shows return array assembly 700 coupled to introducer 702. As shown in FIG. 7B, introducer 702 includes a handle 730 that is positioned distal of return array assembly 700. Lesion generating catheter 704 extends distally from introducer 702 through vasculature 732, and a distal end 734 of lesion generating catheter 702 is positioned in a left atrium 736. Distal end 734 includes at least one lesion generating electrode 738. In this embodiment, elongated electrode 716 is positioned in a cardiac blood supply pool, but not in the heart itself.

During operation, PFA is accomplished by applying electric fields between at least one lesion generating electrode 738 and elongated electrode 716.

Notably, elongated electrode 716 has a much larger electrode surface area than at least one lesion generating electrode 738. Accordingly, when electric fields are applied, current densities at elongated electrode 716 are much lower than current densities at least one lesion generating electrode 738. This results in lesions being generated at at least one lesion generating electrode 738 (i.e., in left atrium 736), but not at elongated electrode 716.

As described above, various embodiments of a return array including a relatively large electrode surface area are possible. In some embodiments, if a long array of return electrodes is utilized, a higher resistance may occur at a distal end of the array, resulting in potential generation of a shadow lesion at the distal end. To avoid this, an electrode at the distal end of the array may be shorted to the other electrodes in the array. However, it is still desirable to have a somewhat high resistance at the distal end. Accordingly, a resistive component may be used to facilitate evenly distributing current across the return array. For example, a coil electrode may be used, with a pitch of the coil electrode set to achieve the desired resistance. Alternatively or additionally, a semi-resistant coating may be applied to one or more electrodes, electrodes may be made of different materials (e.g., higher conductive materials for more proximal electrodes, higher resistivity materials for more distal electrodes), electrodes may have different surface areas, and/or electrodes may have different shapes (e.g., holes and/or etch-outs may be defined through more distal electrodes) to selectively modify the resistivity of the electrodes, such that current is evenly distributed across the return array.

As noted above, in some embodiments, PFA is accomplished by applying voltages between a lesion targeting array and a plurality of return patches distributed on the exterior of the patient.

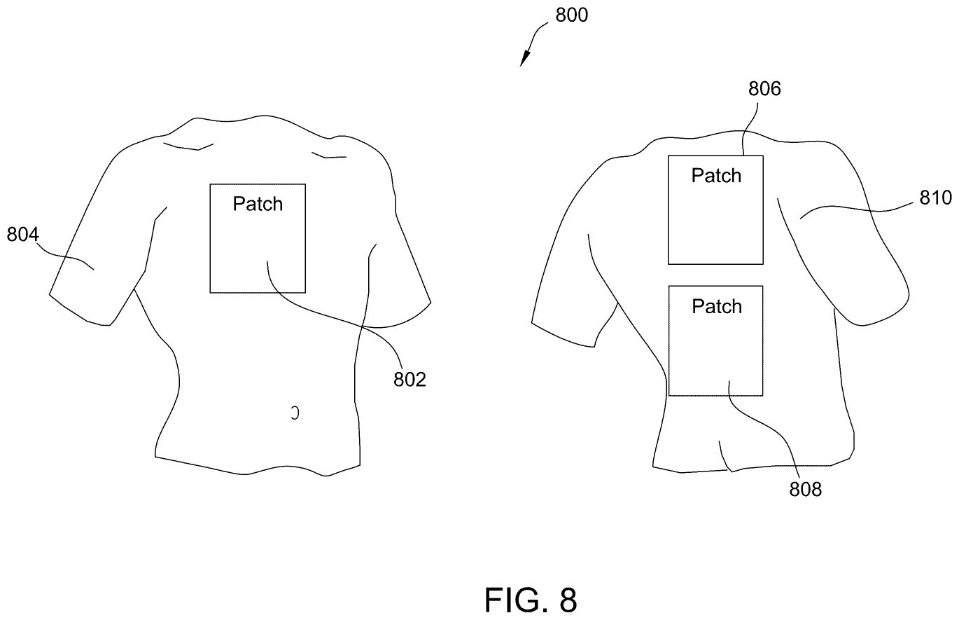

For example, FIG. 8 is a schematic diagram of one embodiment of a patch arrangement 800. Patch arrangement 800 includes a first patch 802 on a front 804 of the patient, and a second patch 806 and a third patch 808 on a back 810 of the patient. As shown in FIG. 8, the patches are centered on the back 810 and front 804 of the patient. Of course, those of skill in the art will appreciate that patch arrangement 800 is only an example, and that other suitable configurations may be used.

Each of patches 802, 806, and 808 may be relatively large to achieve a more distributed effect. Alternatively, two or more patches 802, 806, and 808 may be activated simultaneously to function as a larger effective electrode to achieve the distributed effect. Those of skill in the art will appreciate that patches 802, 806, and 808 may have any suitable size and shape. For example, patches may be circular, rectangular, or square. Further, patches may have a width and a height each between 2 to 7 inches (50.8 to 177.8 millimeters), for example. This results in a patch surface are between 4 and 49 inches.

Having a distributed patch network reduces the total energy density at each patch 802, 806, and 808, which facilitates reducing skeletal muscle recruitment during PFA therapy. In some embodiments, patches 802, 806, and 808 are activated iteratively. For example, for a first period of time, voltage is applied between a lesion targeting array (not shown) and first patch 802, for a second period of time subsequent to the first period of time, voltage is applied between the lesion targeting array and second patch 806, and for a third period of time subsequent to the second period of time, voltage is applied between the lesion targeting array and third patch 908. Activating patches 802, 806, and 808 in series reduces the total joules seen at each patch, reduces the electrical impulse seen at each patch, and reduces the overall current through the patch network, while distributing voltages drop across patches 802, 806, and 808. Again, this facilitates reducing skeletal muscle recruitment. Notably, distributing current across patches located in various positions on the body facilitates reducing patient movement

Those of skill in the art will appreciate that the various embodiments of catheter assemblies described herein may be implemented independently from one another or in any suitable combination.

The embodiments described herein provide a catheter assembly. The catheter assembly includes a catheter including at least one lesion generating electrode, the at least one lesion generating electrode configured to be positioned within a patient, and at least one return array configured to be positioned within the patient and remote from the at least one lesion generating electrode, the at least one return array including at least one return electrode, wherein the catheter assembly is configured to apply energy between i) the at least one lesion generating electrode and ii) a return patch and the at least one return electrode to generate lesions proximate the at least one lesion generating electrode.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

1. A catheter assembly comprising:
a catheter comprising at least one lesion generating electrode, the at least one lesion generating electrode configured to be positioned within a patient; and
at least one return array configured to be positioned within the patient and remote from the at least one lesion generating electrode, the at least one return array comprising at least one return electrode, wherein the catheter assembly is configured to apply energy between i) the at least one lesion generating electrode and ii) a return patch and the at least one return electrode to generate lesions proximate the at least one lesion generating electrode.

2. The catheter assembly of claim 1, wherein the at least one return electrode has a larger electrode surface area than the at least one lesion generating electrode.

3. The catheter assembly of claim 1, further comprising at least one additional catheter, wherein the at least one additional catheter comprises the at least one return array.

4. The catheter assembly of claim 1, wherein the at least one return array comprises a selectively inflatable balloon surrounding the at least one return electrode.

5. The catheter assembly of claim 1, wherein the at least one return array comprises a plurality of insulated struts surrounding the at least one return electrode.

6. The catheter assembly of claim 1, wherein, to apply energy, the catheter assembly is configured to apply at least one of: radio frequency energy; or pulsed field ablation energy.

7. The catheter assembly of claim 1, wherein the at least one return electrode comprises a wrapped electrode that is helically wrapped around a catheter body.

8. The catheter assembly of claim 1, wherein the at least one return electrode comprises a plurality of ring electrodes.

9. The catheter assembly of claim 1, wherein the at least one return electrode comprises a single elongated electrode.

10. The catheter assembly of claim 1, further comprising a sleeve configured to be slid over an introducer, wherein the sleeve comprises the at least one return array.

11. An ablation system comprising:
a generator; and
a catheter assembly coupled to the generator, the catheter assembly comprising:
a catheter comprising at least one lesion generating electrode, the at least one lesion generating electrode configured to be positioned within a patient; and
at least one return array configured to be positioned within the patient and remote from the at least one lesion generating electrode, the at least one return array comprising at least one return electrode, wherein the generator is configured to apply energy between i) the at least one lesion generating electrode and ii) a return patch and the at least one return electrode to generate lesions proximate the at least one lesion generating electrode.

12. The ablation system of claim 11, wherein the at least one return electrode has a larger electrode surface area than the at least one lesion generating electrode.

13. The ablation system of claim 11, wherein the catheter assembly further comprises at least one additional catheter, wherein the at least one additional catheter comprises the at least one return array.

14. The ablation system of claim 11, wherein the at least one return array is configured to be positioned in a blood stream of the patient.

15. The ablation system of claim 11, wherein the catheter assembly further comprises a resistive component configured to facilitate evenly distributing current over the at least one return array.

16-23. (canceled)

24. The ablation system of claim 11, wherein the at least one return array comprises at least one of: a selectively inflatable balloon surrounding the at least one return electrode; or a plurality of insulated struts surrounding the at least one return electrode.

25. The ablation system of claim 11, wherein, to apply energy, the catheter assembly is configured to apply at least one of: radio frequency energy; or pulsed field ablation energy.

26. The ablation system of claim 11, wherein the at least one return electrode comprises a wrapped electrode that is helically wrapped around a catheter body.

27. The ablation system of claim 11, wherein the at least one return electrode comprises one of: a plurality of ring electrodes; or a single elongated electrode.

28. The ablation system of claim 11, wherein the catheter assembly further comprises a sleeve configured to be slid over an introducer, wherein the sleeve comprises the at least one return array.
